(19) United States
(12) Patent Application Publication
(10) Pub. No.: US 2011/0105337 A1
(43) Pub. Date: May 5, 2011

(54) METHOD OF DISCOVERING AND ANALYZING SECRETED BIOMARKERS OF DISEASE FROM SOLID TISSUE

(76) Inventor: David B. Krizman, Gaithersburg, MD (US)

(21) Appl. No.: 12/666,288

(22) PCT Filed: Jun. 23, 2008
(86) PCT No.: PCT/US08/67937

§ 371 Date: Nov. 29, 2010

Related U.S. Application Data
(60) Provisional application No. 60/945,662, filed on Jun. 22, 2007.

(51) Int. Cl. C12Q 1/00; C40B 20/00

(52) U.S. Cl. 506/2; 435/40.52

(57) ABSTRACT

The current invention provides a method for discovering protein biomarkers of disease for use in diagnostic assays of bodily fluids by determining differential expression patterns of proteins secreted or released directly by normal and diseased epithelial cells into glandular lumens. Determining those secreted or released proteins directly from the glandular lumen of diseased and normal solid tissue would lead to a catalogue of proteins that have a high degree of probability to be present in various bodily fluids in persons with specific diseases. This would prove useful as a means to diagnose specific conditions and diseases by simply assaying easily acquired bodily fluids. Past efforts to discover such diagnostic/screening markers in bodily fluids have proven difficult at best due to overriding complexity of the proteins within bodily fluids. This invention is a method of discovering those biomarkers in a more focused and less complex protein subpopulation, namely the secreted or released proteins present in glandular lumens.

### FIELD OF THE INVENTION

The invention provides compositions and methods for detecting of disease biomarkers in biological samples, including samples prepared from formalin-fixed tissue specimens.

### BACKGROUND OF THE INVENTION

Trained pathologists and histotechnologists perform molecular analyses on cancer tissue to determine the expression of specific molecular biomarkers. Information derived from such analyses is valuable prognostic tool. For instance, a number of different protein biomarkers are associated with specific stages or grades of cancer and are relied upon to guide therapies. The current method of immunohistochemistry (IHC) is very capable of analyzing biomarkers that are highly predictive of a specific stage or grade of cancers. However, in order to perform such a clinical analysis on a tissue sample, it is necessary to procure tissue from a cancer patient either by invasive surgical or by biopsy procedures. These methods can be disruptive and potentially harmful to the patient. Methods to detect the presence of the same clinically relevant biomarkers in patient samples collected by minimally or non-invasive methods are very advantageous. Such patient samples are readily collected by minimally or non-invasive methods from easily-obtained bodily fluids such as whole blood, plasma, serum, saliva, tears, urine, and sweat. However, very few known biomarkers that are detected in tissue can be detected and assayed in bodily fluids such as serum and urine. This is primarily due to a disconnect between knowledge of clinically useful biomarkers that are detected in tissue and those that are detected in bodily fluids.

### SUMMARY OF THE INVENTION

In general, the present invention relates to detection of diseases such as cancer by analysis of biomarkers contained in biological samples containing luminal proteins. Specifically, the invention provides new methods to identify and characterize disease biomarkers from glandular luminal tissue. Advantageously, the biological samples are obtained from histologically processed samples, including formalin-fixed tissue, by manipulations including the generation of Liquid Tissue preparations.

In a first aspect, the invention provides a method of identifying a disease biomarker by providing a biological sample having material obtained from a glandular lumen, which contains a protein present within the glandular lumen, and identifying the protein in the biological sample, thereby identifying the disease biomarker. The material obtained from the glandular lumen is from a tissue section, such as might be obtained by the method of tissue microdissection. In certain embodiments, the biological sample is obtained from a mammalian subject suffering from, or at risk of, developing the disease. In other embodiments, the method provides for the identification of two or more proteins in the biological sample, thereby generating a disease protein profile.

Optionally, the disease protein profile is compared with a control protein profile containing two or more proteins identified in a control biological sample that includes a protein (e.g., a soluble protein) present within a glandular lumen obtained from a mammalian subject not suffering from or not known to be at risk of developing the disease. In some embodiments, the disease protein profile is obtained from two or more biological samples from two or more glandular lumens, which can be obtained from a single subject or from two or more subjects. The control protein profile can be obtained from two or more biological samples from two or more glandular lumens, which can be obtained from a single subject or two or more subjects. In other embodiments, the disease protein profile contains two or more proteins not measurably detectable in the control protein profile. In some embodiments, the disease protein profile and the control protein profile are obtained from the same tissue or organ type. The biological sample or control biological sample optionally are prepared using Liquid Tissue® reagents and protocols. In some embodiments the biological sample is obtained from an animal model of a human disease. The biological sample and/or the control biological sample may include material obtained from an epithelial layer adjacent to the glandular lumen.

In a second aspect, the invention provides a method of identifying a disease biomarker by providing a first biological sample containing a protein present within a glandular lumen, where the biological sample contains material obtained from the glandular lumen, and a second biological sample that contains material obtained from an epithelial layer adjacent to the glandular lumen, identifying a biomarker protein present in the first biological sample, and comparing the amount of the biomarker protein present in the first biological sample and in the second biological sample.

The method provides for identification of the protein by mass spectrometry, e.g., RPLC-MS/MS, LC-ESI-MS, LC-ESI-MS/MS, LC-nanospray-MS, LC-nanospray-MS/MS, MALDI-TOF, MALDI-TOF/TOF, SELDI-TOF, or SELDI-TOF/TOF. Alternatively, the protein is identified using a protein array and an immuno-based assay.

Preferably, the sample material comprises histopathologically processed tissue, fresh tissue, or frozen tissue. Histopathologically processed tissue can be formalin-fixed tissue or cells, formalin-fixed/paraffin embedded (FFPE) tissue or cells, FFPE tissue blocks and cells from said blocks, FFPE tissue blocks and cells from diseased and normal tissue, or FFPE tissue blocks and cells from biopsies obtained surgically. Frozen tissue is frozen by freezing in air or liquid freezing. The biological sample can include a multi-use biomolecule lysate (e.g., a Liquid Tissue® preparation) prepared by the steps of heating a composition containing the biological sample and a reaction buffer at a temperature and a time sufficient to negatively affect protein cross-linking in the biological sample, and treating the resulting composition with an effective amount of a proteolytic enzyme for a time sufficient to disrupt the tissue and cellular structure of the biological sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

### DESCRIPTION OF THE DRAWINGS AND FIGURES

The present invention may be further appreciated with reference to the appended drawing sheets wherein:

FIG. 1 is a schematic illustration demonstrating one embodiment of the invention for analyzing proteins obtained from glandular lumens using mass spectrometry.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

One of the most important factors to be understood by a clinician in order to provide optimal treatment of a disease is the diagnosis of disease status. For example, is a tumor benign or invasive? This frequently involves determining the expression status of a biomarker of the disease. As used herein, a disease biomarker includes a particular protein or form of a protein preferentially present in, or absent from, diseased cells as compared to normal cells, which indicates the stage/grade or the severity of the disease, and under certain conditions predicts the course of disease. Disease biomarkers also include non-protein organic molecules produced in vivo. Advantageous biomarkers are those proteins that also provide an indication of the choice of therapy. Preferably, such a biomarker is linked to a specific disease or group of diseases. It is desirable to interrogate the biomarker in the most accurate and least invasive setting possible, meaning that analysis of the presence or absence of the biomarker in a given tissue is performed not in the tissue itself, but in biological samples, particularly bodily fluids, which are typically amenable to being sampled. Minimally invasive or non-invasive collection provides fluids, including but not limited to, plasma, serum, whole blood, saliva, spinal fluid, tears, and urine. However, a current limitation to biomarker analysis is the lack of biomarkers that can be assayed in bodily fluids.

The present invention solves this problem by providing methods for the discovery and analysis of secreted biomarkers, generally peptide fragments of luminal proteins, which are useful in detecting and characterizing or staging solid tissue diseases and disorders. The term “protein” is intended to include peptides and polypeptides that have amino acid sequences which are less than but contained within the sequences of full-length proteins. A peptide can include from two to about fifty amino acids (e.g., 5, 10, 15, 20, 30, 40 or 50 amino acids). The proteins secreted or released by diseased cells into glandular lumens and collecting ducts are directed into bodily fluids such as urine, saliva, and blood. As used herein, a “glandular lumen” includes an inner open space or cavity within a tissue, that is generally surrounded by secretory epithelial cells. A glandular lumen collects secreted or released proteins (or peptide fragments thereof) and moves this protein population, often through a series of lumens, into collecting ducts. The protein complexity of these bodily fluids is high so that discovering and identifying disease biomarkers in these biological samples represents a substantial challenge. In addition, the complexity of cellular proteins often prevents efficient identification of peptide sequences from these cellular proteins that are useful as disease biomarkers.

Prior to the present invention, the art lacked a method by which proteins (or peptide fragments thereof) secreted or released into glandular lumens and connecting ducts could be isolated and identified. Moreover, prior methods have focused on analysis of whole tissue or regions of tissue without recognizing and studying specific structures, such as glandular lumens and connecting ducts that are present in these large regions of tissue. Also, prior to the present invention, laser-based tissue microdissection generally has been performed on whole cells that make up specific features in the tissue itself. Microdissection has not been utilized to specifically remove and procure proteins residing within glandular lumens that are not present at detectable levels within the whole cells.

This invention is directed in part to a novel approach to discovering and identifying biomarkers of disease directly from proteins contained within glandular lumens present in standard cut tissue sections. First, a biological sample is obtained that contains a protein present within a glandular lumen. Next, the luminal protein is identified by one or more means; often, the protein is modified, such as by proteolytic cleavage, prior to the identification step, in order to detect peptide fragments instead of the full-length protein itself. Validation of the identified proteins as biomarkers is performed, for example, by comparison with a biological sample obtained from normal (i.e., not diseased) tissue. As now demonstrated herein, biomarkers preferentially found in the glandular lumens and connecting ducts of all types of organs and tissue are identified from both frozen and formalin-fixed tissue samples. These biomarkers are novel polypeptides useful in detecting and staging disease, including cancer.

### Biological Samples

The invention provides biological samples containing a protein present within a glandular lumen. Proteins found in glandular lumens include extracellular proteins, secreted or released proteins, intracellular proteins, membrane-bound proteins, nuclear proteins, cytoplasmic proteins, and organelle proteins. Specifically advantageous glandular lumen proteins include extracellular proteins and secreted proteins. Obtaining a sample solely from the glandular lumen allows one to create a profile (or “archive”) of the proteins expressed in both the normal lumen and the diseased lumen. Often, the proteinaceous material is “trapped” in the lumen when the tissue sample is fixed, such as by freezing or chemical means. Samples are tissues obtained from, e.g., biopsies, diagnostic surgical pathology or cytology. Any biological sample that has been removed from an organism that has solid organ tissue is a sample according to the present invention. The biological sample is derived from a glandular lumen from a normal or a diseased solid tissue sample, which can include fresh tissue, frozen tissue, fresh frozen tissue, or histopathologically processed tissue.

Preferably, the tissue sample is histopathologically processed, such as by formalin fixation of tissues or cells, formalin fixation and paraffin embedding of tissues or cells, or formalin fixation and/or paraffin embedding of solid tumor tissue. Histopathological processing typically occurs through the use of a formalin fixative. Formalin is used widely because it is relatively inexpensive, easy to handle, and once the formalin-fixed sample is embedded in paraffin the sample is stored easily. Additionally, formalin is often the fixative of choice because it preserves both tissue structure and cellular morphology. Although the exact mechanism may not be understood fully, without wishing to be bound by theory it is believed that fixation occurs by formalin-induced cross-linking of the proteins within the biological specimen. Due to these protein cross-links, formalin fixation has found wide success in the traditional microscopic analysis of histological sections.

Recently, a novel set of reagents and methods have been developed that allows one skilled in the art to solubilize protein and peptides directly from formalin fixed solid tissue for analysis by advanced assays. (See, U.S. patent application Ser. No. 10/796,288, filed on Mar. 10, 2004, the contents of which are incorporated herein by reference in their entirety) Prior to this discovery, few experimental techniques were available for analysis of proteins and peptides from histopathologically processed solid tissue samples, including histopathologically processed solid tissue and histopathologically processed biopsies obtained from solid tissue, due to the insolubility of the protein as a result of the formalin fixation, and these prior techniques contained substantial limitations. Previously, formalin fixation rendered formalin-fixed pathologic tissue, including histopathologically processed solid organ tissue and histopathologically processed biopsies obtained from solid organ tissue, of little value for many of the powerful analysis methods that have been developed in recent years such as mass spectrometry and protein arrays.

Formalin fixation for processing and storage of surgically removed solid tissue and biopsies, including those solid tissues and biopsies derived from tumors, is well known by those skilled in the art. For nearly the last one hundred years, solid body tissue has been routinely fixed in formalin or formalin fixed/paraffin wax-embedded (FFPE) blocks. The overwhelming majority of hospitals and pathology laboratories, in the course of diagnostic surgical and anatomic pathology, process all solid tissue with formalin prior to use for many diagnostic tests.

In order to utilize histologically processed disease and normal tissue, the invention provides for the creation of a biological sample containing a soluble protein/peptide lysate from glandular lumens within histopathologically processed diseased and normal solid tissue. The biological sample is generated from a glandular lumen within the histopathologically processed solid diseased and/or normal tissue or biopsy from such tissue, e.g., a tissue or cellular sample. The biological sample is amenable to subsequent manipulations, including extractions, isolations, further solubilizations, fractionations, dilutions and storage.

Preferred methods for obtaining luminal biological samples include tissue microdissection, which specifically remove proteinaceous material contained in the lumen. Examples of other methods of obtaining a biological sample from a tissue include, but are not limited to, using a manual core punch, tissue punch, laser-based tissue microdissection and other techniques that are well known in the arts. The actual size of the obtained biological sample is not important as long as there is a sufficient amount to perform the chosen assay.

### Tissue Microdissection of Biological Samples

Tissue microdissection includes several different technologies that are designed to procure specific cell populations in a highly enriched form, generally directly from thin tissue sections on glass slides. The different technologies for achieving microdissection can be applied to tissue that has been preserved in a frozen state or to tissue that has been chemically preserved, such as that which is achieved by formalin fixation. One methodology is performed by placing the thin tissue section on a plastic-coated glass slide and then placing that glass slide directly above a laser mounted on a microscope. The area of cells to be separated from the rest of the tissue sample is visualized by the microscope and then completely outlined by either repeated or continuous use of the laser to cut the plastic and the tissue attached to the plastic. The portion of the plastic that encompasses the cells is then moved into a collection container, such as with a single shot of the laser whereby the plastic, along with the attached cells, flies up into the collection tube. Alternatively, if the plastic-coated glass slide contacting the tissue section is turned upside down and placed underneath a laser mounted on a microscope, by virtue of gravity the plastic and the cells attached to the outlined plastic fall into a collection tube following laser treatment of the periphery.

In another method, a thin tissue section is placed on a standard glass slide and placed underneath a microscope-mounted laser. The cells of interest are then visually identified and a cap with a thin photo-reactive film is placed directly on top of the tissue and the laser is activated. By virtue of the laser hitting the photo-reactive film, the film is activated such that the cells directly underneath attach to the underside of the film. The film is then removed with the cells and other material of interest attached thereto and placed into a collection tube for further processing. In still another iteration of tissue microdissection, a thin tissue section is placed on a glass slide with a thin energy transfer coating. This glass slide is then placed underneath a microscope-mounted laser and the cells of interest are microscopically visualized. The laser is then activated to strike the energy transfer coating, which transforms UV light energy into heat, thus causing the cells and material of interest to travel into a collection tube.

### Analysis of Luminal Biological Samples

The present invention provides for biochemical analysis of cellular components (including proteins or peptides thereof, lipids, carbohydrates and other materials) referred to herein as “analytes,” within glandular lumens of a solid diseased and/or normal tissue. An analyte includes any protein or peptide derived therefrom, or any other cellular component, that is capable of being detected by a detection means known in the art, such as mass spectrometry, Western blotting, immuno-based assays, or a protein array. Preferably, the analyte also is capable of being quantified such that the levels of the analyte in a diseased tissue can be compared with levels in a corresponding tissue derived from an individual not suffering from the disease. Analytes include individual proteins, groups or families of proteins, or multiple members of groups of proteins. When the biomolecule of interest is a peptide, the peptide is directly derived from a previously intact protein from the extract in a soluble liquid form and the peptide and/or collection of peptides derive from and is representative of the total protein content of the glandular lumen procured from the starting diseased or normal solid tissue sample. As with proteins, any resulting peptide extract from a starting protein extract can be placed in any number of peptide/protein identification, analysis, and expression assays including but not limited to mass spectrometry, Western blots, immuno-based assays, and protein arrays.

The analysis of the proteins contained within a normal or diseased glandular lumen is encompassed by the term “proteomics”, which most generally refers to the large scale study of proteins. For instance, all proteomic technologies rely on the ability to separate a complex mixture so that individual proteins are more easily processed with other techniques. Proteomics also involves the identification of specific proteins. For instance, identification may involve low-throughput sequencing through Edman degradation. Higher-throughput proteomic techniques are based on mass spectrometry, commonly peptide mass fingerprinting, or de novo repeat detection sequencing on instruments capable of more than one round of mass spectrometry. Antibody-based assays can also be used, but usually are specific to one sequence motif. Other aspects of proteomic studies involve the quantification of protein, the sequence analysis of proteins, the structure of proteins, the interaction of proteins, the modification of proteins and the mapping of the location of proteins in the cell.

A preferred method of luminal protein analysis is mass spectrometry. Examples of mass spectrometry instrument formats include but are not limited to LC-ESI-MS, LC-ESI-MS/MS, LC-nanospray-MS, LC-nanospray-MS/MS, MALDI-TOF, MALDI-TOF/TOF, SELDI-TOF, and SELDI-TOF/TOF.

The LC-ESI-MS/MS mass spectrometry method of global proteomic profiling involves preparing a protein sample from cells of interest using one of a variety of protein sample preparation protocols. The protein preparation is then digested into peptides with a common protease, e.g., trypsin, and the resulting digestate is injected into a liquid chromatography column. The separation is based on characteristics such as size and charge of individual peptides. As separate fractions elute from the column they are injected by electron spray ionization into a mass spectrometer for peptide identification. Based on the ion trapping and bombardment physics of a mass spectrometry, the primary amino acid structures of peptides are determined and a list of peptides is generated. The masses of the peptides of the unknown proteins in the sample are compared to the theoretical peptide masses of each protein encoded in the genome to generate a list of protein identifications. In general, profiling by mass spectrometry using LC-ESI-MS/MS provides for up to 3,000 protein identifications in a two hour chromatography separation.

Another mass spectrometry method is MALDI, where tryptic digests are placed onto a metal plate and allowed to dry together with a chemical matrix. A laser is used to activate the matrix, which has the effect of shooting the matrix containing the peptides directly into a mass spectrometer. The same methodology of peptide bombardment then takes place to provide for amino acid sequencing of peptides and identification of peptides and the proteins from which these peptides are derived. In this way, mass spectrometry analysis provides a detailed identification of proteins present in the starting protein preparation. MALDI mass spectrometry can identify about 200 proteins from a single complex biological sample.

Comparison of Luminal Proteins from Diseased and Normal Tissue

In the present invention, each biological sample containing soluble proteins and peptides as generated herein directly reflects the status of those proteins in the glandular lumen from the diseased solid tissue sample, or from a corresponding normal tissue sample. Thus, in accordance with the invention, the proteins detected in such glandular lumen comprise a protein profile that is then compared with the proteins discovered within the glandular lumen of a normal solid tissue sample in order to identify differentially expressed proteins that may be protein biomarkers of disease and that can provide for a diagnostic assay of bodily fluid.

The best method for comparing diseased proteins to normal proteins is to utilize a computer program to compare datasets to each other, and to ascertain the differences and commonalties between these two datasets. This is accomplished, for example, by detecting one or more peptides through tandem mass spectrometry for each of the samples and creating a list or index of peptides contained in each sample. The peptides from each list then are used to identify the proteins from which they are derived. Generally, this identification is carried out using one of many commercially available computer programs known in the art, such as SEQUEST (Eng et al., “An approach to correlate tandem mass spectral data of peptides with amino acid sequences in the protein database,” Journal of American Society of Mass Spectrometry 1994, 5(11):976-989) and MASCOT (Perkins et al., “Probability-based protein identification by searching database using mass spectrometry data, “Electrophoresis 1999, 20(18):3551-3567). The information regarding identified proteins is analyzed using a software program such as Microsoft Access in order to develop protein expression datasets. These protein identifications form the foundation for lists of proteins that are present in diseased glandular lumen and absent in normal glandular lumen or that are present at different levels in the two samples. It is these proteins that are useful as disease biomarkers, and which have a high probability to be present in bodily fluids such as serum. Assaying for these proteins in serum provides a way to diagnose a disease using minimally invasive methods for patient sample collection.

The present invention is not limited to the particular methodologies, protocols, constructs, formulae and reagents described but further include those known to the skilled artisan. It is also to be understood that the terminology used herein is for the purpose of describing particular embodiments only, and is not intended to limit the scope of the present invention.

### Example 1

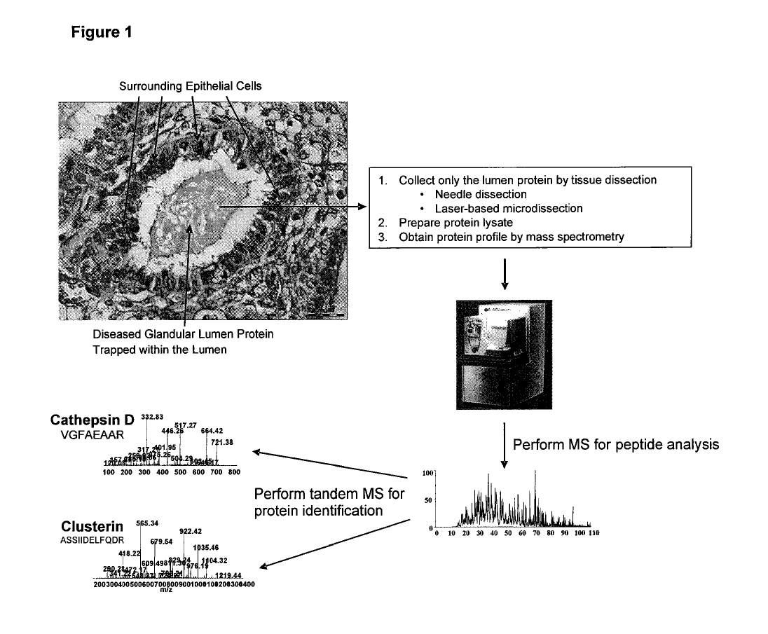

Table 1 is a list of proteins found commonly expressed both in uterine fluid as discovered from a literature survey and in soluble protein lysate from the glandular lumen shown in FIG. 1. Of note is that all of the proteins previously shown to be secreted by endometrial cells through a survey of the published literature between 1986 and 2003 are also identified in the protein profile of the endometrial glandular lumen.

### Example 2

Table 2 shows results from an analysis that determined what types of proteins are present in the glandular lumen protein profile and compares that to the types of proteins from a protein profile determined by mass spectrometry of protein from epithelial cells. Note that the highest percentage of protein in the glandular lumen sample represents proteins that are extracellular associated and membrane bound, whereas a smaller percentage of the total protein is of intracellular origin. The highest percentage of protein in the epithelial cell sample is of intracellular origin. These results indicate that the lumen does in fact contain predominantly secreted extracellular protein and that this invention is advantageous for specifically identifying secreted protein by analysis of protein present within organ and glandular lumens.

### Example 3

FIG. 4 shows the complete list of proteins identified by mass spectrometry analysis of a soluble protein lysate derived from the organ/glandular lumen from a single histopathologically processed solid tumor tissue sample of endometrial origin shown in FIG. 1.

TABLE 1
Galectin-3 binding protein precursor (Lectin galactoside-binding soluble 3 binding protein)
Gelsolin precursor (Actin-depolymerizing factor)(ADF)(Brevin)(AGEL)
Glycodelin precursor (GD)(Pregnancy-associated endometrial alpha-2 globulin)
Heat shock cognate 71 kDa protein (Heat shock 70 kDa protein 8)
Heat shock protein HSP 90-beta (HSP 84)(HSP 90)
Heat shock-related 70 kDa protein 2 (Heat shock 70 kDa protein 2)
Insulin-like growth factor binding protein 7 precursor (IGFBP-7)(IBP-7)
Lipocalin-1 precursor (Von Ebner gland protein)(VEG protein) (Tear prealbumin)
Serotransferrin precursor (Transferrin)(Siderophilin)(Beta-1-metal binding globulin)

TABLE 2

| | Cancer Cell | Lumen Protein |
|---|---|---|
| Extracellular Proteins | 9.31% | 37.88% |
| Intracellular Proteins | 67.37% | 15.15% |
| Membrane Proteins | 21.23% | 37.88% |

TABLE 3
14-3-3 protein zeta/delta (Protein kinase C inhibitor protein 1) (KCIP-1)
60S ribosomal protein L13 (Breast basic conserved protein 1)
Actin, aortic smooth muscle (Alpha-actin-2)
Actin, cytoplasmic 1 (Beta-actin)
ALB protein
Alpha-1 catenin (Cadherin-associated protein) (Alpha E-catenin)
Alpha-1-antitrypsin precursor (Alpha-1 protease inhibitor) (Alpha-1-antiproteinase)
Alpha-enolase, lung specific (EC 4.2.1.11) (2-phospho-D-glycerate hydro-lyase) (Non-neural enolase) (NNE)
Alpha-tocopherol transfer protein (Alpha-TTP)
Ankyrin-3 (ANK-3) (Ankyrin G)
Annexin A1 (Annexin I) (Lipocortin I) (Calpactin II) (Chromobindin-9) (p35) (Phospholipase A2 inhibitory
Annexin A2 (Annexin II) (Lipocortin II) (Calpactin I heavy chain) (Chromobindin-8) (p36) (Protein I) (Placental)
Annexin A5 (Annexin V) (Lipocortin V) (Endonexin II) (Calphobindin I) (CBP-I) (Placental anticoagulant protein)
Annexin IV variant (Fragment)
AP-3 complex subunit mu-1 (Adapter-related protein complex 3 mu-1 subunit) (Mu-adaptin 3A) (AP-3 adapter)
Apolipoprotein A-I precursor (Apo-AI) (ApoA-I) [Contains: Apolipoprotein A-I(1-242)]
Apoptotic protease-activating factor 1 (Apaf-1)
ARAR9245
AT rich interactive domain 4B (RBP1-like)
ATP synthase gamma chain, mitochondrial precursor
Beta-globin gene from a thalassemia patient, complete cds
C1orf109 protein
Calmodulin (CaM)
cAMP-specific 3′,5′-cyclic phosphodiesterase 4A (EC 3.1.4.17) (DPDE2) (PDE46)
Carbamoyl-phosphate synthase [ammonia], mitochondrial precursor (EC 6.3.4.16) (Carbamoyl-phosphate)
CDNA FLJ34378 fis, clone FEBRA2018051
CDNA FLJ35778 fis, clone TESTI2005378
Cell division cycle associated 1
Centaurin-delta 3 (Cnt-d3) (Arf-GAP, Rho-GAP, ankyrin repeat and pleckstrin homology domain-containing
Ceruloplasmin precursor (EC 1.16.3.1) (Ferroxidase)
Chloride anion exchanger (DRA protein) (Down-regulated in adenoma) (Solute carrier family 26 member 3)
Chromodomain helicase-DNA-binding protein 5 (EC 3.6.1.-) (ATP-dependent helicase CHD5) (CHD-5)
Clusterin precursor (Complement-associated protein SP-40,40) (Complement cytolysis inhibitor) (CLI)
Coatomer subunit delta (Delta-coat protein) (Delta-COP) (Archain)
Coiled-coil domain-containing protein 13
Collagen alpha-1(I) chain precursor
Collagen alpha-2(I) chain precursor
Complement C3 precursor [Contains: Complement C3 beta chain; Complement C3 alpha chain; C3a
Complement C4-A precursor (Acidic complement C4) [Contains: Complement C4 beta chain; Complement
Cytosolic nonspecific dipeptidase (Glutamate carboxypeptidase-like protein 1) (CNDP dipeptidase 2)
Dehydrogenase E1 and transketolase domain containing protein 1
Deleted in malignant brain tumors 1
Dermcidin precursor (Preproteolysin) [Contains: Survival-promoting peptide; DCD-1]
Desmin
DnaJ protein
Elongation factor 1-alpha 2 (EF-1-alpha-2) (Elongation factor 1 A-2) (eEF1A-2) (Statin S1)
Ezrin (p81) (Cytovillin) (Villin-2)
Ferritin light polypeptide variant
Fibrillin-2 precursor
Fibrinogen alpha chain precursor [Contains: Fibrinopeptide A]
Fibulin-1 precursor
Fructose-bisphosphate aldolase A (EC 4.1.2.13) (Muscle-type aldolase) (Lung cancer antigen NY-LU-1)
Galectin-3 binding protein precursor (Lectin galactoside-binding soluble 3 binding protein) (Mac-2 binding)
Gelsolin precursor (Actin-depolymerizing factor) (ADF) (Brevin) (AGEL)
Glutamate [NMDA] receptor subunit epsilon 4 precursor (N-methyl D-aspartate receptor subtype 2D) (NR2D)
Glyceraldehyde-3-phosphate dehydrogenase (EC 1.2.1.12) (GAPDH)
Glycodelin precursor (GD) (Pregnancy-associated endometrial alpha-2 globulin) (PEG) (PAEG) (Placental)
HBS1-like protein (ERFS)
Heat shock cognate 71 kDa protein (Heat shock 70 kDa protein 8)
Heat shock protein HSP 90-beta (HSP 84) (HSP 90)
Heat shock-related 70 kDa protein 2 (Heat shock 70 kDa protein 2)
Hemoglobin delta subunit (Hemoglobin delta chain) (Delta-globin)
Heterogeneous nuclear ribonucleoproteins A2/B1 (hnRNP A2/hnRNP B1)
Histone H2A type 1 (H2A.2)
Histone H4
Homeobox protein BarH-like 1
Homeobox protein Hox-A5 (Hox-1C)
Hornerin
Hsp89-alpha-delta-N
Hypothetical protein
Hypothetical protein DKFZp686C1522
Hypothetical protein DKFZp686J1372
Hpothetical protein DKFZp762N1910 (Fragment)
Hypothetical protein DKFZp781H1112
Hypothetical protein FLJ22833
Hypothetical protein FLJ35808
Hypothetical protein MGC42367
Hypothetical protein MGC9850 (Polymerase (RNA) I polypeptide C)
Hypothetical protein SH3PX3 (Hypothetical protein DKFZp666B159)
Ig gamma-1 chain C region
Ig kappa chain V-II region Cum
Ig kappa chain V-III region SIE
IgG receptor FcRn large subunit p51 precursor (FcRn) (Neonatal Fc receptor) (IgG Fc fragment receptor
IGHA1 protein
IGHM protein
IGKC protein
IGLC2 protein
Immunoglobulin J chain
Immunoglobulin superfamily member 21 precursor
Insulin receptor substrate 2 insertion mutant (Fragment)
Insulin-like growth factor binding protein 7 precursor (IGFBP-7) (IBP-7) (IGF-binding protein 7) (MAC25)
Interleukin-12 beta chain precursor (IL-12B) (IL-12 p40) (Cytotoxic lymphocyte maturation factor 40 kDa
Isocitrate dehydrogenase [NADP] cytoplasmic (EC 1.1.1.42) (Oxalosuccinate decarboxylase) (IDH)
Keratin, type I cytoskeletal 10 (Cytokeratin-10) (CK-10) (Keratin-10) (K10)
Keratin, type I cytoskeletal 12 (Cytokeratin-12) (CK-12) (Keratin-12) (K12)
Keratin, type I cytoskeletal 14 (Cytokeratin-14) (CK-14) (Keratin-14) (K14)
Keratin, type I cytoskeletal 16 (Cytokeratin-16) (CK-16) (Keratin-16) (K16)
Keratin, type I cytoskeletal 19 (Cytokeratin-19) (CK-19) (Keratin-19) (K19)
Keratin, type I cytoskeletal 9 (Cytokeratin-9) (CK-9) (Keratin-9) (K9)
Keratin, type II cytoskeletal 1 (Cytokeratin-1) (CK-1) (Keratin-1) (K1) (67 kDa cytokeratin) (Hair alpha protein)
Keratin, type II cytoskeletal 2 epidermal (Cytokeratin-2e) (K2e) (CK 2e)
Keratin, type II cytoskeletal 5 (Cytokeratin-5) (CK-5) (Keratin-5) (K5) (58 kDa cytokeratin)
Keratin, type II cytoskeletal 6A (Cytokeratin-6A) (CK 6A) (K6a keratin)
Keratin, type II cytoskeletal 8 (Cytokeratin-8) (CK-8) (Keraton-8) (K8)
Kinesin-like protein KIF14
Leucine-rich repeat kinase 1
Lipocalin-1 precursor (Von Ebner gland protein) (VEG protein) (Tear prealbumin) (TP) (Tear lipocalin) (Tlc)
Lipophilin-B precursor (Secretoglobin family 1D member 2)
L-lactate dehydrogenase A chain (EC 1.1.1.27) (LDH-A) (LDH muscle subunit) (LDH-M)
Maltase-glucoamylase, intestinal [Includes: Maltase (EC 3.2.1.20) (Alpha-glucosidase); Glucoamylase
MELL1 protein
Microtubule-associated protein (Fragment)
Mitogen-activated protein kinase kinase kinase 1 (EC 2.7.1.37) (MAPK/ERK kinase kinase 1) (MEK kinase 1)
Mitogen-activated protein kinase kinase kinase 10 (EC 2.7.1.37) (Mixed lineage kinase 2) (Protein kinase)
Multidrug resistance-associated protein 6 (ATP-binding cassette sub-family C member 6) (Anthracycline)
Myosin-10 (Myosin heavy chain, nonmuscle IIb) (Nonmuscle myosin heavy chain IIb) (NMMHC II-b)
Myosin-9 (Myosin heavy chain, nonmuscle IIa) (Nonmuscle myosin heavy chain IIa) (NMMHC II-a)
Nebulin-related anchoring protein
Neurosecretory protein VGF precursor
Niban-like protein (Meg-3)
Novel protein similar to Ankyrin repeat domain protein 18A (LOC441424) (Fragment)
Nuclear receptor-interacting protein 1 (Nuclear factor RIP140) (Receptor-interacting protein 140)
Orexin receptor type 1 (Ox1r) (Hypocretin receptor type 1)
Osteopontin precursor (Bone sialoprotein-1) (Secreted phosphoprotein 1) (SPP-1) (Urinary stone protein)
Otoancorin precursor
Peroxiredoxin-6 (EC 1.11.1.15) (Antioxidant protein 2) (1-Cys peroxiredoxin) (1-Cys PRX)
PIWIL3 protein
Plasma protease C1 inhibitor precursor (C1 Inh) (C1Inh)
Plasminogen activator inhibitor 1 precursor (PAI-1) (Endothelial plasminogen activator inhibitor) (PAI)
Probable G-protein coupled receptor 97 precursor (G-protein coupled receptor PGR26)
Probable helicase senataxin (EC 3.6.1.-) (SEN1 homolog)
Programmed cell death protein 8, mitochondrial precursor (Apoptosis-inducing factor)
Protection of telomeres 1 (hPot1) (POT1-like telomere end-binding protein)
Protein C20orf121
Protein CXorf38
Protein KIAA0494
Prothymosin alpha [Contains: Thymosin alpha-1]
Receptor for advanced glycosylation end-products deletion exon2-6 variant
Recombining binding protein suppressor of hairless-like protein (Transcription factor RBP-L)
RGD, leucine-rich repeat, tropomodulin and proline-rich containing protein (Fragment)
Rho-associated protein kinase 1 (EC 2.7.1.37) (Rho-associated, coiled-coil containing protein kinase 1)
Serine/threonine-protein kinase PLK4 (EC 2.7.1.37) (Polo-like kinase 4) (PLK-4) (Serine/threonine-protein)
Serotransferrin precursor (Transferrin) (Siderophilin) (Beta-1-metal binding globulin)
Shroom-related protein
Similar to CG3714 gene product
Sodium channel protein type X alpha subunit (Voltage-gated sodium channel alpha subunit Nav1.8)
Sodium/hydrogen exchanger 5 (Na(+)/H(+) exchanger 5) (NHE-5) (Solute carrier family 9 member 5)
Solute carrier family 22 member 11 variant (Fragment)
Spectrin alpha chain, brain (Spectrin, non-erythroid alpha chain) (Alpha-II spectrin) (Fodrin alpha chain)
Spermatid perinuclear RNA-binding protein
Spermatogenesis associated 18 homolog
Tetratricopeptide repeat protein 12 (TPR repeat protein 12)
THAP domain-containing protein 2
Thioredoxin (ATL-derived factor) (ADF) (Surface associated sulphydryl protein) (SASP)
Thioredoxin reductase 2, mitochondrial precursor (EC 1.8.1.9) (TR3) (TR-beta) (Selenoprotein Z) (SelZ)
Transcription elongation factor B polypeptide 1 (RNA polymerase II transcription factor SIII subunit C)
Transforming acidic coiled-coil-containing protein 1 (Taxin 1) (Gastric cancer antigen Ga55)
Transgelin-2 (SM22-alpha homolog)
Transitional endoplasmic reticulum ATPase (TER ATPase) (15S Mg(2+)-ATPase p97 subunit) (Valosin)
Tricarboxylate transport protein, mitochondrial precursor (Citrate transport protein) (CTP) (Tricarboxylate)
Trinucleotide repeat-containing gene 6A protein (CAG repeat protein 26) (Glycin-tryptophan protein)
Triosephosphate isomerase (EC 5.3.1.1) (TIM) (Triose-phosphate isomerase)
Tropomyosin alpha-3 chain (Tropomyosin-3) (Tropomyosin gamma) (hTM5)
Tropomyosin beta chain (Tropomyosin 2) (Beta-tropomyosin)
Tubulin alpha-1 chain (Alpha-tubulin 1) (Testis-specific alpha-tubulin) (Tubulin H2-alpha)
Tubulin alpha-2 chain (Alpha-tubulin 2)
Tubulin beta-2 chain
Tubulin beta-2C chain (Tubulin beta-2 chain)
Ubiquitin
Ubiquitin-activating enzyme E1 (A1S9 protein)
Versican core protein precursor (Large fibroblast proteoglycan) (Chondroitin sulfate proteoglycan core
Vimentin
Vitamin D-binding protein precursor (DBP) (Group-specific component) (Gc-globulin) (VDB)
Vitronectin precursor (Serum spreading factor) (S-protein) (V75) [Contains: Vitronectin V65 subunit)
Whirlin (Autosomal recessive deafness type 31 protein)
Zinc finger protein 679

Unless defined otherwise, all technical and scientific terms used herein have the same meaning as commonly understood to one of ordinary skill in the art to which this invention belongs. Any methods, devices, and materials similar or equivalent to those described herein can be used in the practice or testing of the invention. All publications and patents mentioned herein are incorporated herein by reference.

What is claimed is:

1. A method of identifying a disease biomarker, comprising the steps of:
a) providing a biological sample comprising a substanially pure material obtained from a glandular lumen obtained from a mammalian subject having or at risk of developing the disease; and
b) identifying a protein in the biological sample;
thereby identifying the disease biomarker.

2. The method of claim 1, wherein the material is obtained from the glandular lumen from a tissue section by the method of tissue microdissection.

3. The method of claim 1, wherein the mammalian subject has cancer.

4. The method of claim 1, comprising identifying two or more proteins in the biological sample, thereby generating a disease protein profile.

5. The method of claim 4, wherein the disease protein profile is compared with a control protein profile comprising two or more proteins identified in a control biological sample comprising a substanially pure material obtained from a glandular lumen obtained from a mammalian subject not having or not at risk of developing the disease.

6. The method of claim 5, wherein the disease protein profile contains two or more proteins not measurably detectable in the control protein profile.

7. The method of claim 5, wherein the disease protein profile contains two or more proteins at measurably different levels than in the control protein profile.

8. The method of claim 4, wherein the disease protein profile is obtained from two or more biological samples from two or more glandular lumens.

9. The method of claim 8, wherein the two or more glandular lumens are obtained from a plurality of mammalian subjects.

10. The method of claim 5, wherein the control protein profile is obtained from two or more biological samples from two or more glandular lumens.

11. The method of claim 8, wherein the two or more glandular lumens are obtained from a plurality of mammalian subjects.

12. The method of claim 5, wherein the disease protein profile and the control protein profile are obtained from the same tissue or organ type.

13. The method of claim 1, wherein the biological sample is obtained from an animal model of a human disease.

14. The method of claim 1, wherein the protein is identified by mass spectrometry.

15. The method of claim 13, wherein said mass spectrometry is selected from the group consisting of RPLC-MS/MS, LC-ESI-MS, LC-ESI-MS/MS, LC-nanospray-MS, LC-nanospray-MS/MS, MALDI-TOF, MALDI-TOF/TOF, SELDI-TOF, and SELDI-TOF/TOF.

16. The method of claim 1, wherein the material comprises histopathologically processed tissue, fresh tissue, or frozen tissue.

17. The method of claim 16, wherein the histopathologically processed tissue is selected from the group consisting of formalin-fixed tissue or cells, formalin-fixed/paraffin embedded (FFPE) tissue or cells, FFPE tissue blocks and cells from said blocks, FFPE tissue blocks and cells from diseased and normal tissue, and FFPE tissue blocks and cells from biopsies obtained surgically.

18. The method of claim 4, wherein the frozen tissue is frozen by freezing in air or liquid freezing.

19. The method of claim 1, wherein the biological sample comprises a multi-use biomolecule lysate prepared by the steps of:
a) heating a composition comprising the biological sample and a reaction buffer at a temperature and a time sufficient to negatively affect protein cross-linking in the biological sample, and
b) treating the resulting composition with an effective amount of a proteolytic enzyme for a time sufficient to disrupt the tissue and cellular structure of said biological sample.

20. The method of claim 18, wherein the biological sample is prepared using Liquid Tissue® reagents and protocols.

21. The method of claim 5, wherein the control biological sample is prepared using Liquid Tissue® reagents and protocols.

22. The method of claim 1, wherein the protein is identified using a protein array and an immuno-based assay.

23. The method of claim 4, wherein the disease protein profile is compared with a control protein profile comprising two or more proteins identified in a control biological sample comprising a substanially pure material obtained from epithelial cells surrounding a glandular lumen, wherein the material is obtained from a mammalian subject not having or not at risk of developing the disease.

24. The method of claim 1, wherein the protein is a soluble protein.

25. The method of claim 1, wherein the protein contains one or more soluble peptides.

26. The method of claim 1, wherein the protein is selected from the group consisting of proteins listed in Tables 1 and 3.

27. A method of identifying a disease biomarker, comprising the steps of:
a) providing a first biological sample comprising a protein present within a glandular lumen, wherein the biological sample comprises material obtained from the glandular lumen;
b) providing a second biological sample comprises material obtained from an epithelial layer adjacent to the glandular lumen;
c) identifying a biomarker protein present in the first biological sample; and
d) comparing the amount of the biomarker protein present in the first biological sample and in the second biological sample,
thereby identifying the disease biomarker.
